(19) United States
(12) Patent Application Publication
(10) Pub. No.: US 2026/0158531 A1
(43) Pub. Date: Jun. 11, 2026

(54) NOVEL MICROBE HAVING PLASTIC DECOMPOSITION ACTIVITY AND USE THEREOF

(71) Applicant: INDUSTRY FOUNDATION OF CHONNAM NATIONAL UNIVERSITY, Gwangju (KR)

(72) Inventors: Soo Jin YEOM, Gwangju (KR); Chul Ho YUN, Sejong-si (KR); Won Seok CHI, Gwangju (KR); Seung Do YUN, Gwangju (KR)

(73) Assignee: INDUSTRY FOUNDATION OF CHONNAM NATIONAL UNIVERSITY, Gwangju (KR)

(21) Appl. No.: 18/686,248

(22) PCT Filed: Aug. 25, 2022
(86) PCT No.: PCT/KR2022/012749

§ 371 Date: Feb. 23, 2024

(30) Foreign Application Priority Data
Aug. 26, 2021 (KR) 10-2021-0113277

(51) Int. Cl. B09B 3/60; B09B 101/75; B09B 101/77; C12N 1/205; C12R 1/07

(52) U.S. Cl. CPC B09B 3/60; C12N 1/205; B09B 2101/75; B09B 2101/77; C12R 2001/075

(57) ABSTRACT

The present invention relates to a Bacillus thuringiensis JNU01 (accession number: KCTC 14427BP) strain having a plastic degradation activity. The Bacillus thuringiensis JNU01 strain according to the present invention is able to degrade plastic when cultured in a medium comprising the plastic, and thus may be used in a pretreatment process for plastic recycling.

### TECHNICAL FIELD

This application claims the benefit of the filing date of Korean Patent Application No. 10-2021-0113277, filed with the Korea Intellectual Property Office on Aug. 26, 2021, the entire contents of which are incorporated in the present invention.

The present invention relates to a novel microorganism having plastic degradation activity and the use thereof.

### BACKGROUND ART

Plastic refers to organic-based polymeric materials and mixtures thereof that can be molded by heat or pressure. Plastic is light yet strong, can be easily shaped into any shape, can hold anything, and even has a long lifespan and is inexpensive. Thus, plastic is used in a variety of applications, including films, synthetic fibers, bottles, tubes, and toys, which are widely used in their daily lives, as well as high-heat-resistance and high-strength materials. As the use of plastic has increased, plastic production has also increased by about 4% on average per year, reaching about 15.488 million metric tons of plastic production in 2016, and has continued to increase to this day.

However, plastic, which provides convenience in life, is not easily degraded and is attracting attention as the major cause of soil and marine environment pollution, and plastic production and consumption continue to increase. Thus, finding a way to degrade plastic is currently an urgent challenge.

### DISCLOSURE

### Technical Problem

An object of one aspect is to provide a Bacillus thuringiensis JNU01 (accession number: KCTC 14427BP) strain having plastic degradation activity.

An object of another aspect is to provide a method for degrading plastic comprising a step of culturing the Bacillus thuringiensis JNU01 strain in a medium containing the plastic.

### Technical Solution

One aspect provides a Bacillus thuringiensis JNU01 (accession number: KCTC 14427BP) strain having plastic degradation activity.

According to one embodiment, the plastic may be one or more of polyethylene terephthalate (PET), polyvinyl chloride (PVC), polystyrene (PS), polypropylene (PP), and polyethylene (PE). For example, the plastic may be polyethylene (PE).

According to one embodiment, the Bacillus thuringiensis JNU01 strain may be capable of growing using the plastic as a carbon source.

In one example, it was found that the Bacillus thuringiensis JNU01 strain grew when the concentration of polyethylene (PE) was 30 mg/mL to 50 mg/mL.

Another aspect provides a method for degrading plastic comprising a step of culturing the Bacillus thuringiensis JNU01 strain in a medium containing the plastic.

According to one embodiment, the plastic may be one or more of polyethylene terephthalate (PET), polyvinyl chloride (PVC), polystyrene (PS), polypropylene (PP), and polyethylene (PE).

Although the medium containing the plastic may further contain known substances necessary for culturing the strain, in addition to the plastic described above, it may not contain any carbon sources other than the plastic when considering the purpose of the present invention.

As described above, the strain of the present invention is able to degrade plastic, especially polyethylene, and thus when the strain is cultured in a medium containing plastic, the strain located on the surface of polyethylene can form pores on the surface of polyethylene by its metabolic activity.

### Advantageous Effects

The Bacillus thuringiensis JNU01 strain according to the present invention is able to degrade plastic when cultured in a medium containing the plastic, and thus it may be used in a pretreatment process for plastic recycling.

### BRIEF DESCRIPTION OF DRAWINGS

FIG. 1a depicts photographs showing the location of soil sample collection and the process of collecting the soil sample to isolate and identify a plastic degrading strain.

FIG. 1b schematically shows a process of selecting the strain from the collected sample.

FIG. 2a depicts photographs showing the results of an experiment for examining whether a microorganism degrades polyethylene (PE) in liquid media. Specifically, the portion indicated by (a) shows a liquid medium containing only polyethylene, the portion indicated by (b) shows a liquid medium containing polyethylene and the microorganism, and the portion indicated by (c) shows a liquid medium containing only the microorganism.

FIG. 2b shows data obtained by measuring the O.D. value to examine whether the strain in the medium indicated by (b) grew before and after the experiment shown in FIG. 2a.

FIG. 3 shows the phylogenetic tree of the Bacillus thuringiensis JNU01 strain isolated as a plastic degrading microorganism.

FIG. 4 shows data obtained by measuring the growth rate of the Bacillus thuringiensis JNU01 strain depending on the concentration of polyethylene.

FIG. 5 shows NMR data for polyethylene (marked as “Control”) not treated with the Bacillus thuringiensis JNU01 strain and polyethylene (marked as “B. thuringiensis”) degraded by treatment with the Bacillus thuringiensis JNU01 strain.

FIG. 6 shows FT-IR analysis data for polyethylene (marked as “Control”) not treated with the Bacillus thuringiensis JNU01 strain and polyethylene (marked as “B. thuringiensis”) degraded by treatment with the Bacillus thuringiensis JNU01 strain. In addition, the portions indicated by (a) to (f) at the bottom of FIG. 6 show comparative data obtained by placing the FT-IR data portions of Control and B. thuringiensis on the same line.

FIG. 7 shows scanning electron microscope (SEM) images of a polyethylene film (marked as “PE film) not treated with the Bacillus thuringiensis JNU01 strain and a polyethylene film (marked as “B. thuringiensis”) degraded by treatment with the Bacillus thuringiensis JNU01 strain. In FIG. 7, the portion indicated by (A) and the portion indicated by (D) are, respectively, 2,000× and 10,000× magnification images of the surface of the polyethylene film (marked as “PE film) not treated with the Bacillus thuringiensis JNU01 strain, and the portion indicated by (B), the portion indicated by (C), the portion indicated by (E), and the portion indicated by (F) are 2,000× and 10,000× magnification images of the surface of the polyethylene film (marked as “B. thuringiensis”) degraded by treatment with the Bacillus thuringiensis JNU01 strain. In FIG. 7, the portion indicated by (B) and the portion indicated by (C) are images of different positions of the same polyethylene film, the portion indicated by (E) is a 10,000× magnification image of the position corresponding to the portion indicated by (B), and the portion indicated by (F) is a 10,000× magnification image of the position corresponding to the portion indicated by (C).

### BEST MODE

Throughout the present specification, it is to be understood that when any part is referred to as “comprising” any component, it does not exclude other components, but may further comprise other components, unless otherwise specified.

Hereinafter, the present invention will be described in more detail.

One aspect provides a Bacillus thuringiensis JNU01 (accession number: KCTC 14427BP) strain having plastic degradation activity.

According to one embodiment, the plastic may be one or more of polyethylene terephthalate (PET), polyvinyl chloride (PVC), polystyrene (PS), polypropylene (PP), and polyethylene (PE). For example, the plastic may be polyethylene (PE).

According to one embodiment, the Bacillus thuringiensis JNU01 strain may be capable of growing using the plastic as a carbon source.

In one Example, it was found that the Bacillus thuringiensis JNU01 strain grew when the concentration of polyethylene (PE) was 30 mg/mL to 50 mg/mL.

Another aspect provides a method for degrading plastic comprising a step of culturing the Bacillus thuringiensis JNU01 strain in a medium containing the plastic.

According to one embodiment, the plastic may be one or more of polyethylene terephthalate (PET), polyvinyl chloride (PVC), polystyrene (PS), polypropylene (PP), and polyethylene (PE).

Although the medium containing the plastic may further contain known substances necessary for culturing the strain, in addition to the plastic described above, it may not contain any carbon sources other than the plastic when considering the purpose of the present invention.

### MODE FOR INVENTION

Hereinafter, one or more embodiments will be described in more detail by way of examples. However, these examples are intended to illustrate one or more embodiments and the scope of the present invention is not limited to these examples.

### Example 1. Experimental Methods

### 1.1. Purchase of Media, Purchase of Polyethylene, and Preparation of Polyethylene Powder and Film

Nutrient broth (NB) medium, Luria Bertani (LB) broth, yeast extract, and basal salt medium (BSM) were purchased from MB Cell (2F, Kisan B/D, 11, Yangjaecheon-ro 31-gil, Seocho-gu, Seoul), and a trace element solution (per liter, 21.8 mg CoCl₂·6H₂O, 21.6 mg NiCl₂·6H₂O, 24.6 mg CuSO₄·5H₂O, 1.62 mg FeCl₃·6 H₂O, 0.78 g CaCl₂) and 14.7 mg MnCl₂·4 H₂O) was prepared. Polyethylene (average M_{w}: about 4,000 by GPC, average Mₙ: about 1,700 by GPC, CAS Number 9002-88-4) was purchased from Sigma-Aldrich (St. Louis, MO, USA) and prepared into powder and films.

1.2. Isolation of Microorganisms from Soil and Examination of Cell Growth in Polyethylene-Containing Medium

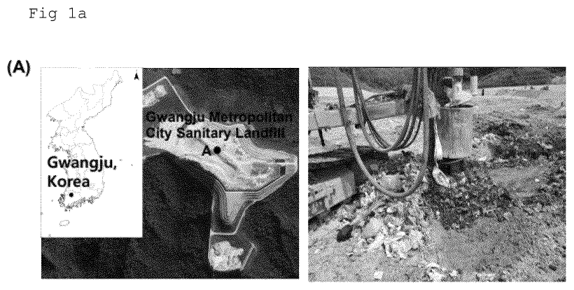

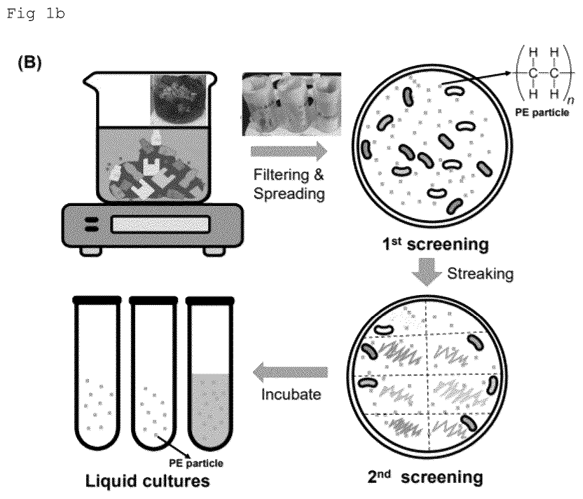

Soil was collected from 15M underground at the Gwangju Environmental Corporation Metropolitan Sanitation Landfill (160 Dodong-gil, Nam-gu, Gwangju, site A in FIG. 1a: 35°05′06′9″N, 126°53′32′8″E). Microorganisms were isolated from the collected soil in the same manner as shown in FIG. 1b. Specifically, about 100 g of the collected soil was mixed with 600 mL of PBS buffer in a 2 L beaker and filtered through Whatman paper (pore size: 11 μm). The filtered solution was spread on BSM solid medium plates containing 1 g/L of polyethylene powder and incubated at 28° C. for 7 days. Colonies cultured on the BSM solid media containing polyethylene were streaked on BSM solid medium plates containing 1 g/L of polyethylene, and whether the colonies would grow in the BSM solid medium plates containing polyethylene was checked again. Then, 43 colonies grown on the BSM solid medium plates containing 1 g/L of polyethylene powder were inoculated into liquid nutrient media and cultured at 28° C. and 200 rpm for 15 hours. The cultured bacteria were centrifuged at 3,800 rpm for 20 minutes and then washed with BSM liquid medium. The bacteria washed with BSM liquid medium were inoculated into a BSM liquid medium containing 1 g/L of polyethylene powder and cultured at 28° C. and 200 rpm for 50 days. Also, the optical density (OD) was measured at 10-day intervals.

### 1.3. Identification of Bacterial Strains

43 single colonies were obtained from the cultured bacteria by streaking onto NB solid medium plates (3.0 g beef extract, 5.0 g peptone, 15.0 g agar in 1 L of deionized water, pH 6.8). DNA of the Bacillus thuringiensis JNU01 strain selected from the 43 colonies was extracted using a HiGene™ Genomic DNA Prep Kit (BIOFACT, Daejeon, South Korea). To determine the bacterial species, 16S rRNA gene sequencing was performed using universal primers 27F (AGAGTTTGATCCTGGCTCAG) and 1492R (GGTTACCTTGTTACGACTT) (Solgent, Daejeon, South Korea).

### 1.4. Growth of Microorganisms in Polyethylene-Containing Medium

The identified Bacillus thuringiensis JNU01 strain was inoculated into a liquid nutrient medium and cultured in a shaking incubator overnight at 28° C. and 200 rpm. Then, the strain was centrifuged at 3,800 rpm for 20 minutes and washed with BSM liquid medium. Then, the strain was inoculated into a flask containing polyethylene powder, trace element solution, and BSM liquid medium and cultured at 28° C. and 200 rpm for 30 days. For comparison, a flask containing only polyethylene, trace element solution, and BSM liquid medium without the Bacillus thuringiensis JNU01 strain was used as a control. Finally, culture of the strain was performed using polyethylene powder concentrations of 10, 30 and 50 mg/ml. The optical density (OD) was measured using a UV-Vis spectrophotometer (SHIMADZU, Kyoto, Japan) at 5-day intervals.

1.5. Analysis of Chemical structure, Physical Properties and Morphology of Polyethylene

The selected bacteria were inoculated into liquid media containing polyethylene powder and polyethylene film (0.6 cm×0.4 cm) and cultured at 28° C. and 200 rpm for 30 days. After culture, the polyethylene powder and polyethylene film were washed with 2% SDS solution for 4 hours, and then washed three times with 10 mL of deionized water and MeOH. The washed polyethylene powder and film were completely dried in a vacuum oven at 60° C. and analyzed by FT-IR, NMR, and SEM. 1H NMR analysis was performed using a Nuclear Magnetic Resonance Spectrometer (Unity INOVA 500, 500 MHz) at the Korea Basic Science Institute (KBSI, Gwangju, South Korea) at 25° C. NMR sample solutions were prepared as 0.5% (w/v) solutions using CDCl₃ solvent. The chemical shifts were measured in parts per million (ppm) based on the residual peak of CDCl₃ (7.26 ppm). FT-IR (Fourier-transform infrared spectroscopy, IRAffinity-1S, Shimadzu, Kyoto, Japan) was used to detect the functional groups of polyethylene samples in the wavelength range of 4000-500 cm⁻¹ with a resolution of 4 cm⁻¹. The surface morphologies of the polyethylene films were observed using a FE-SEM (filed emission scanning electron microscope, Hitachi S-4800) with an accelerating voltage of 15 kV at the Center for Scientific Instrument, Chosun University, Gwangju. Before FE-SEM measurement, a conductive layer was directly formed on the surface of the polyethylene film using platinum raw material at 20 mA for 100 seconds, thereby enabling FE-SEM measurement of the polyethylene polymer sample through the platinum coating.

### Example 2. Experimental Results

### 2.1. Isolation and Identification of Bacterial Strains

In order to select polyethylene-degrading strains, as shown in FIG. 1a, soil samples were collected from 15M underground at the Gwangju Environmental Corporation Metropolitan Sanitation Landfill (160 Dodong-gil, Nam-gu, Gwangju, site A in FIG. 1a: 35°05′06′9″N, 126°53′32′8″E). Specifically, as shown in FIG. 1b, polyethylene powder purchased from Sigma-Aldrich was used as an initial selection system. Next, each soil sample was dissolved in PBS buffer and filtered through Whatman paper. Then, the filtrate was spread directly on BSM solid medium plates containing 1 g/L of polyethylene powder as a sole carbon source. After culturing at 28° C. for 7 days, some colonies grew and were streaked on BSM solid medium plates containing 1 g/L of polyethylene powder, and then re-growth of the colonies was confirmed. 43 colonies grown on the BSM solid medium plates containing 1 g/L of polyethylene powder were inoculated into liquid nutrient media and cultured at 28° C. and 200 rpm for 15 hours. The cultured bacteria were centrifuged at 3,800 rpm for 20 minutes and then washed with BSM liquid medium. The washed bacteria were inoculated into BSM liquid medium containing 1 g/L of polyethylene powder, and through this operation, one microbial strain that grew best was selected from among the 43 colonies grown in BSM liquid medium containing 1 g/L of polyethylene powder.

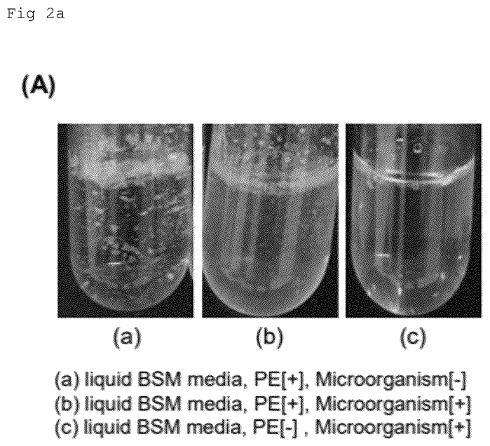

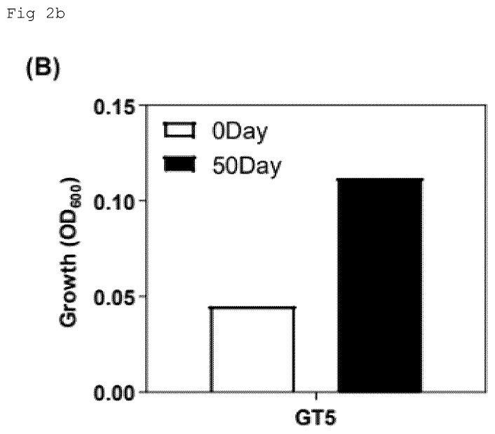

As shown in FIG. 2a, it was found that the size of polyethylene particles in the medium containing the microorganism became smaller than the size of polyethylene particles in the medium containing polyethylene without the microorganism, and the medium containing with only the microorganism without polyethylene was transparent. Also, as shown in FIG. 2b, it was found that the microbial strain was grown successfully in the BSM liquid medium containing 1 g/L of polyethylene powder as the sole carbon source, when measured after 50 days.

As can be seen in Table 1 below, it was found that, based on the 16s rRNA gene sequence, the isolated bacterial strain GT5 was 99% identical to Bacillus thuringiensis.

TABLE 1

| | | | NCBI Reference |
|---|---|---|---|
| | Microorganism | Identity | Sequence |
| GT5 | Bacillus thuringiensis | 99% | NR_114581.1 |

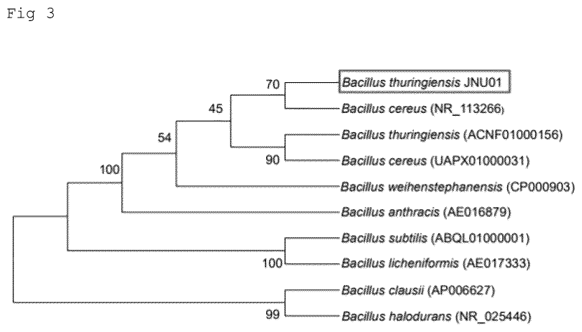

The isolated strain was deposited and named Bacillus thuringiensis JNU01. In addition, as shown in FIG. 3, the phylogenetic tree of the isolated Bacillus thuringiensis JNU01 was confirmed.

2.2. Examination of Growth of Bacillus thuringiensis JNU01 Depending on Polyethylene Concentration

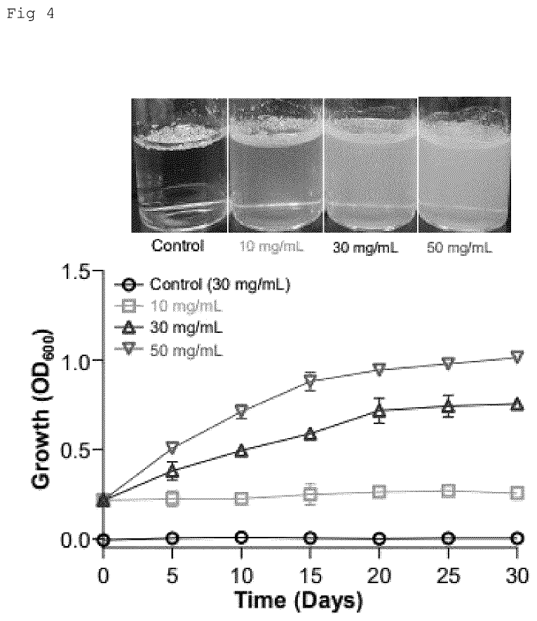

As shown in FIG. 4, in the case of the medium with a polyethylene concentration of 10 mg/mL, there was no significant change in the O.D. value over time. Therefore, it was confirmed that Bacillus thuringiensis JNU01 did not grow in the medium. However, in the medium with a polyethylene concentration of 30 mg/mL and the medium with a polyethylene concentration of 50 mg/mL, the strain reached the exponential growth phase between 1 and 20 days of the experiment, and reached the stationary phase after 20 days of the experiment. Therefore, it was confirmed that Bacillus thuringiensis JNU01 grew in the media with polyethylene concentrations of 30 mg/mL and 50 mg/mL.

### 2.3. NMR Analysis of Polyethylene Powder

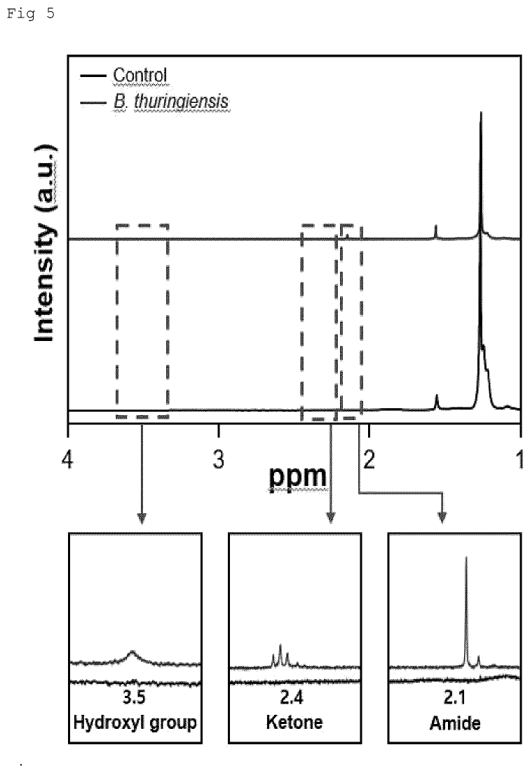

As shown in FIG. 5, as a result of analyzing the polyethylene of the medium containing Bacillus thuringiensis JNU01 after 30 days, it was confirmed that peaks appeared at 2.1 ppm, 2.4 ppm, and 3.5 ppm compared to the control group. The peak at 2.1 ppm means an amide group, the peak at 2.4 ppm means a ketone group, and the peak at 3.5 ppm means a hydroxyl group, suggesting that Bacillus thuringiensis JNU01 degraded polyethylene.

2.4. FT-IR Analysis of Polyethylene Powder

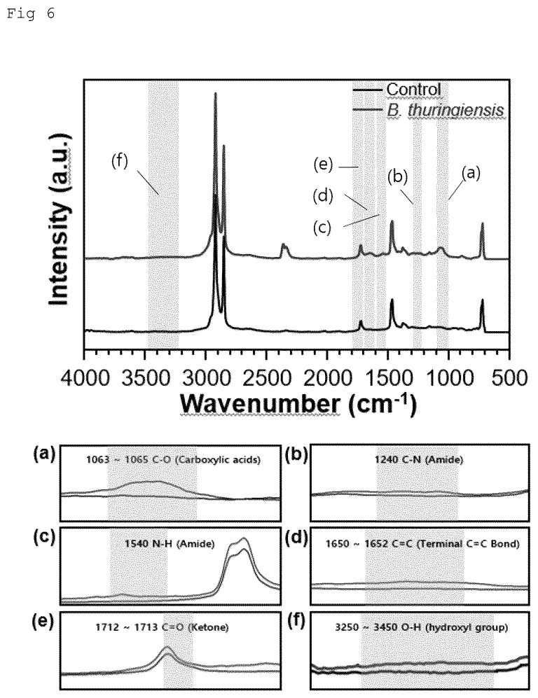

FT-IR was performed to characterize the chemical structure of the polyethylene powder degraded by the strain. As shown in FIG. 6, the FT-IR spectra of the non-degraded polyethylene sample (marked as Control) and the polyethylene sample biologically degraded by culturing Bacillus thuringiensis JNU01 for 30 days were compared. As shown in the portion indicated by (a) in FIG. 6 to the portion indicated by (f) in FIG. 6, the portions from which the chemical structure can be seen are indicated by the same reference line, and thus the non-degraded polyethylene sample (marked as Control) and the polyethylene sample biologically degraded by culturing Bacillus thuringiensis JNU01 for 30 days were compared.

As shown in the portion indicated by (a) in FIG. 6 and the portion indicated by (f) in FIG. 6, it was found that the degraded polyethylene sample further contained carboxylic acid, amide, a terminal C═C bond, ketone, and a hydroxyl group. This shows that, due to biological degradation by Bacillus thuringiensis JNU01, the polyethylene sample may have carboxylic acid, amide, a terminal C═C bond, ketone, and a hydroxyl group.

### 2.5. SEM Images of Polyethylene Films

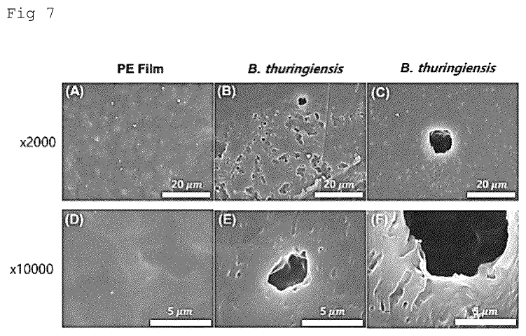

In order to confirm the biological degradation of the polyethylene film by the bacteria, the present inventors measured the FE-SEM images of the polyethylene film not treated with the Bacillus thuringiensis JNU01 strain as shown in portion (A) (2,000×) of FIG. 7 and portion (D) (10,000×) of FIG. 7, and the polyethylene film degraded by treatment with the Bacillus thuringiensis JNU01 strain as shown in portion (B) (2,000×) of FIG. 7, portion (C) (2,000×) of FIG. 7, portion (E) (10,000×) of FIG. 7, and portion (F) (10,000×) of FIG. 7. Portion (B) of FIG. 7 and portion (C) of FIG. 7 show different positions in the polyethylene film degraded by treatment with Bacillus thuringiensis, and portion (E) of FIG. 7 and portion (F) of FIG. 7 are 5-fold enlarged images of portion (B) of FIG. 7 and portion (C) of FIG. 7, respectively. To remove bacteria attached to the polyethylene film degraded by treatment with Bacillus thuringiensis, the polyethylene film was washed with a 2% SDS (sodium dodecyl sulfate) solution for 4 hours and then washed with distilled water and methanol. As shown in portion (A) of FIG. 7 and portion (D) of FIG. 7, the polyethylene film not treated with the Bacillus thuringiensis JNU01 strain was not degraded, and thus had a smooth surface without deformed parts such as holes and cracks. However, as shown in portion (B) of FIG. 7, portion (C) of FIG. 7, portion (E) of FIG. 7, and portion (F) of FIG. 7, it was confirmed that the polyethylene film degraded by treatment with the Bacillus thuringiensis JNU01 strain had significant cracks or pores. As a result of checking the high-magnification FE-SEM images, as shown in portion (E) and portion (F) of FIG. 7, it was confirmed that the polyethylene film degraded by Bacillus thuringiensis had pore structures formed therein. Therefore, it was confirmed that the polyethylene film was degraded by Bacillus thuringiensis.

### ACCESSION NUMBER

Depository authority: Korea Research Institute of Bioscience and Biotechnology
Accession number: KCTC14427BP
Deposit date: Jan. 5, 2021

1. A Bacillus thuringiensis JNU01 (accession number: KCTC 14427BP) strain having plastic degradation activity.

2. The Bacillus thuringiensis JNU01 strain of claim 1, wherein the plastic is one or more of polyethylene terephthalate (PET), polyvinyl chloride (PVC), polystyrene (PS), polypropylene (PP), and polyethylene (PE).

3. The Bacillus thuringiensis JNU01 strain of claim 1, wherein the Bacillus thuringiensis JNU01 strain is able to grow using the plastic as a carbon source.

4. A method for degrading plastic comprising a step of culturing the strain of claim 1 in a medium containing the plastic.

5. The method of claim 4, wherein the plastic is one or more of polyethylene terephthalate (PET), polyvinyl chloride (PVC), polystyrene (PS), polypropylene (PP), and polyethylene (PE).
